(19) United States
(12) Patent Application Publication
(10) Pub. No.: US 2016/0287371 A1
(43) Pub. Date: Oct. 6, 2016

(54) DEVICES AND METHODS FOR DILATING A LUMEN OF A BODY

(71) Applicant: BOSTON SCIENTIFIC SCIMED, INC., Maple Grove, MN (US)

(72) Inventors: PAUL SMITH, Smithfield, RI (US); NIKLAS ANDERSSON, Wayland, MA (US); BRIAN GAFFNEY, Rutland, MA (US); STEVEN E. WALAK, Natick, MA (US); LYNDIA D. PERSONNAT, Billerica, MA (US); JONATHAN ZOLL, Brookline, MA (US); ALISON O. SILBERMAN, Brookline, MA (US); JOHN A. HINGSTON, Framingham, MA (US)

(21) Appl. No.: 15/088,611

(22) Filed: Apr. 1, 2016

Related U.S. Application Data
(60) Provisional application No. 62/142,196, filed on Apr. 2, 2015.

(51) Int. Cl. A61F 2/04; A61F 2/90; A61F 2/88

(52) U.S. Cl. CPC A61F 2/04; A61F 2/88; A61F 2/90; A61F 2002/046

(57) ABSTRACT

Methods and devices for dilating a stricture of a body lumen are disclosed. In some embodiments, a device for dilating a lumen of a body comprises an elongate member having a proximal end and a distal end and an inflatable scaffold member disposed on the distal end of the elongate member, the inflatable scaffold member defining a lumen. In some additional embodiments, the device may further include an inflation member in fluid communication with the lumen of the inflatable scaffold member. Additionally, in some embodiments, the inflatable scaffold member is frangibly connected to the inflation member.

### CROSS-REFERENCE TO RELATED APPLICATIONS

This application claims priority under 35 U.S.C. §119 to U.S. Provisional Application Ser. No. 62/142,196, filed Apr. 2, 2015, the entirety of which is incorporated herein by reference.

### TECHNICAL FIELD

The disclosure is directed to devices and methods for dilating a lumen of a body. More particularly, the disclosure is directed to devices and methods for dilating a lumen of a body and delivery of an inflatable scaffold to the lumen.

### BACKGROUND

Strictures are a narrowing or tightening of a lumen of a body. Some common types of strictures include esophageal strictures affecting the esophagus and blood vessel stenosis which can affect various different blood vessels. Such strictures can cause a variety of problems, for instance, ranging from preventing a patient from getting adequate nutrition to dangerously decreasing the volume of blood flow through a vessel. In some cases, these strictures may be treated using one or more dilation techniques including dilation with bougies, balloon dilation, and implantation of a stent to maintain the opening in the lumen. However, conventional woven metal or fiber stents may be limited in their variety of physical properties.

### BRIEF SUMMARY

The disclosure is directed to several alternative designs, materials and methods of manufacturing medical device structures and assemblies, and uses thereof. In one embodiment, a device for dilating a lumen of a body comprises an elongate member having a proximal end and a distal end, an inflatable scaffold member disposed on the distal end of the elongate member, the inflatable scaffold member defining a lumen, and an inflation member in fluid communication with the lumen of the inflatable scaffold member. In at least some additional embodiments, the inflatable scaffold member is frangibly connected to the inflation member.

Additionally, or alternatively, in the above embodiment, the elongate member is a catheter shaft.

Additionally, or alternatively, in the any of the above embodiments, the device further comprises a balloon member disposed on the proximal end of the catheter shaft, wherein the inflatable scaffold member is disposed around the balloon member.

Additionally, or alternatively, in the any of the above embodiments, the catheter shaft defines at least a guidewire lumen and a first inflation lumen, the first inflation lumen in fluid communication with an interior of the balloon member.

Additionally, or alternatively, in the any of the above embodiments, the catheter shaft defines a second inflation lumen, and wherein the inflation member is in fluid communication with the second inflation lumen.

Additionally, or alternatively, in the any of the above embodiments, the device further comprises a pressure monitor connected to the proximal end of the catheter shaft, the pressure monitor configured to monitor the pressure in the interior of the balloon member.

Additionally, or alternatively, in the any of the above embodiments, the elongate member has one or more recesses for receiving the inflatable stent member.

Additionally, or alternatively, in the any of the above embodiments, the balloon member is non-compliant.

Additionally, or alternatively, in the any of the above embodiments, the inflation member is connected to the balloon member.

Additionally, or alternatively, in the any of the above embodiments, the balloon member has a deflated configuration and an inflated configuration, wherein the frangible connection is configured to break when the balloon member transitions from the inflated configuration to the deflated configuration.

Additionally, or alternatively, in the any of the above embodiments, the inflatable scaffold member has an inflated configuration and a deflated configuration.

Additionally, or alternatively, in the any of the above embodiments, in the inflated configuration, the inflatable scaffold member is radially non-compliant.

Additionally, or alternatively, in the any of the above embodiments, the inflatable scaffold member extends from a proximal end to a distal end along a longitudinal axis, and wherein in the inflated configuration, the inflatable scaffold member is flexible in a longitudinal direction.

Additionally, or alternatively, in the any of the above embodiments, the inflatable scaffold member is connected to the inflation member through a gated port.

Additionally, or alternatively, in the any of the above embodiments, the inflatable scaffold member has a helical coil shape.

Additionally, or alternatively, in the any of the above embodiments, the inflatable scaffold member comprises a plurality of interconnecting conduit members forming a mesh network.

Additionally, or alternatively, in the any of the above embodiments, the inflatable scaffold member comprises a plurality of interconnected rings.

Additionally, or alternatively, in the any of the above embodiments, the inflation member connects to the inflatable scaffold member through a single frangible connection.

Additionally, or alternatively, in the any of the above embodiments, inflation member connects to the inflatable scaffold member through a plurality of frangible connections.

Additionally, or alternatively, in the any of the above embodiments, the inflatable scaffold member is releasably coupled to the elongate member.

Additionally, or alternatively, in the any of the above embodiments, the elongate member has one or more recesses for receiving the inflatable stent member.

Additionally, or alternatively, in the any of the above embodiments, the inflatable scaffold has a first diameter along a first section, and the inflatable scaffold member has a second diameter along a second section, wherein the first diameter is different from the second diameter.

Additionally, or alternatively, in the any of the above embodiments, the elongate member is a semi-rigid member.

Additionally, or alternatively, in the any of the above embodiments, the elongate member tapers toward to the distal end of the elongate member.

This disclosure also relates to an inflatable scaffold device comprising a shaped tubular member defining a lumen, and an inflation member defining an inflation lumen, the inflation lumen in fluid communication with the lumen of the shaped tubular member. In some embodiments, the inflation member is connected to the shaped tubular member by one or more frangible connections.

Additionally, or alternatively, in the above embodiment, the shaped tubular member has a helical coil shape.

Additionally, or alternatively, in the any of the above embodiments, the shaped tubular member comprises a plurality of interconnected rings.

Additionally, or alternatively, in the any of the above embodiments, the shaped tubular member comprises a plurality of interconnecting conduit member forming a mesh network.

Additionally, or alternatively, in the any of the above embodiments, the shaped tubular member has an inflated configuration and a deflated configuration.

Additionally, or alternatively, in the any of the above embodiments, in the inflated configuration, the shaped tubular member is radially non-compliant.

Additionally, or alternatively, in the any of the above embodiments, the shaped tubular member extends from a proximal end to a distal end along a longitudinal axis, and wherein in the inflated configuration, the shaped tubular member is flexible in a longitudinal direction.

Additionally, or alternatively, in the any of the above embodiments, in the inflated configuration, the shaped tubular member is radially non-compliant.

Additionally, or alternatively, in the any of the above embodiments, at least one of the frangible connections is a gated inflation port.

Additionally, or alternatively, in the any of the above embodiments, the inflation member connects to the shaped tubular member through a single frangible connection.

Additionally, or alternatively, in the any of the above embodiments, the inflation member connects to the shaped tubular member through a plurality of frangible connections.

Additionally, or alternatively, in the any of the above embodiments, the shaped tubular has a first diameter along a first section, and the shaped tubular member has a second diameter along a second section, wherein the first diameter is different from the second diameter.

The disclosure also relates to a method for dilating a lumen of a body comprising positioning an elongate member having a proximal end and a distal end within a stricture of a lumen, wherein the elongate member includes an inflatable scaffold member disposed on the distal end of the elongate member, the inflatable scaffold member defining a lumen and frangibly connected to an inflation member and dilating the stricture with the elongate member. In some embodiments, the method further comprises inflating the inflatable scaffold member by delivering inflation media into the lumen of the inflatable scaffold member through the inflation member, and removing the elongate member from within the stricture while leaving the inflatable scaffold member disposed within the stricture.

Additionally, or alternatively, in the any of the above embodiments, inflating the inflatable scaffold member comprises inflating the inflatable scaffold member with one or more solidifying agents.

Additionally, or alternatively, in the any of the above embodiments, the elongate member defines at least a first inflation lumen, the elongate member includes a balloon member disposed on the distal end of the elongate member, an interior of the balloon member is in fluid communication with the first inflation lumen, and dilating the stricture with the elongate member comprises inflating the balloon member.

Additionally, or alternatively, in the any of the above embodiments, removing the elongate member comprises deflating the balloon member.

Additionally, or alternatively, in the any of the above embodiments, the frangible connection is configured to break when the balloon member deflates.

Additionally, or alternatively, in the any of the above embodiments, when the inflatable scaffold member is inflated, the inflatable scaffold member is radially non-compliant.

Additionally, or alternatively, in the any of the above embodiments, the inflatable scaffold member extends from a proximal end to a distal end along a longitudinal axis, and wherein in the inflated configuration, the inflatable scaffold member is flexible in a longitudinal direction.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The aspects of the disclosure may be further understood in consideration of the following detailed description of various embodiments in connection with the accompanying drawings, in which:

FIG. 1 is a schematic illustration of a patient showing an inflatable scaffold dilating a stricture of the patient's esophagus, in accordance with embodiments of the present disclosure;

FIG. 2 is a depiction of an exemplary dilating device, in accordance with embodiments of the present disclosure;

FIG. 3 depicts a partial cross-section of the distal end of the dilating device of FIG. 2;

FIG. 4 is an illustration of an inflatable scaffold, in accordance with embodiments of the present disclosure;

FIG. 5 is an illustration of a distal portion of the inflatable scaffold of FIG. 4 disposed around the dilating device of FIG. 2, in accordance with embodiments of the present disclosure;

FIG. 6 depicts a partial cross-section of the inflatable scaffold of FIG. 4 disposed around dilating device of FIG. 2;

FIGS. 7A-7E are depictions of the inflatable scaffold of FIG. 4 and the dilating device of FIG. 2 disposed within an esophagus of a patient, in accordance with embodiments of the present disclosure;

FIGS. 8A-8D are depictions of example inflatable scaffolds, in accordance with embodiments of the present disclosure;

FIGS. 9A-9B are depictions of a gated connection between an exemplary inflatable scaffold and an inflation member, in accordance with embodiments of the present disclosure;

FIG. 10 is another depiction of a gated connection between an exemplary inflatable scaffold and an inflation member, in accordance with embodiments of the present disclosure;

FIG. 11 is a depiction of an exemplary dilation device including multiple inflation lumens, in accordance with embodiments of the present disclosure;

FIG. 12 depicts an example of a balloon member of a dilation device, in accordance with embodiments of the present disclosure; and

FIG. 13 depicts an alternative example dilation device.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

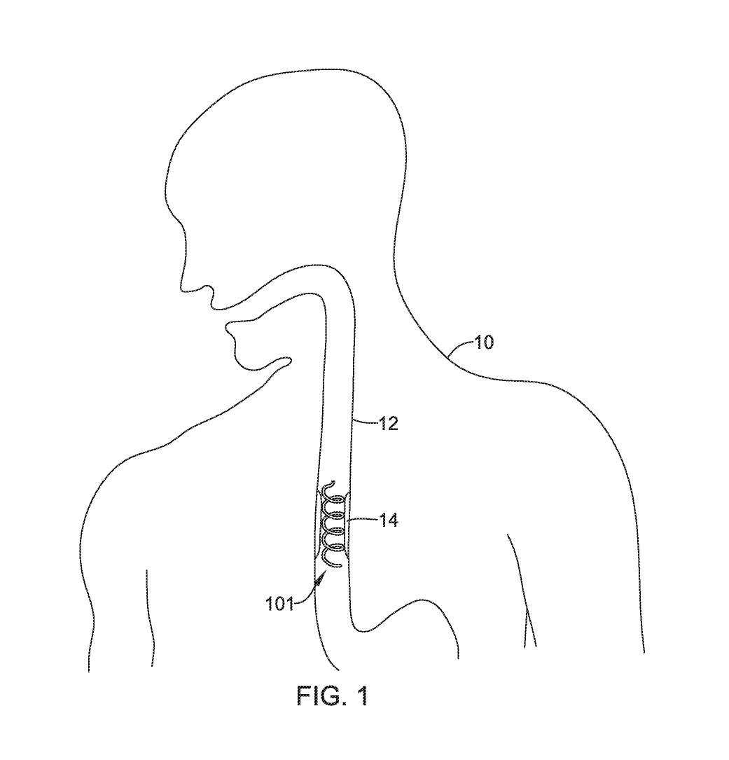

FIG. 1 is a schematic illustration of a torso of a patient 10. Patient 10 includes an esophagus 12 with stricture 14. FIG. 1 also depicts inflatable scaffold member 101 disposed within stricture 14. As will be described herein, inflatable scaffold member 101 may be part of a dilation system for dilating stricture 14. After dilation, inflatable scaffold member 101 may remain within stricture 14 in order to maintain an opening through esophagus 12.

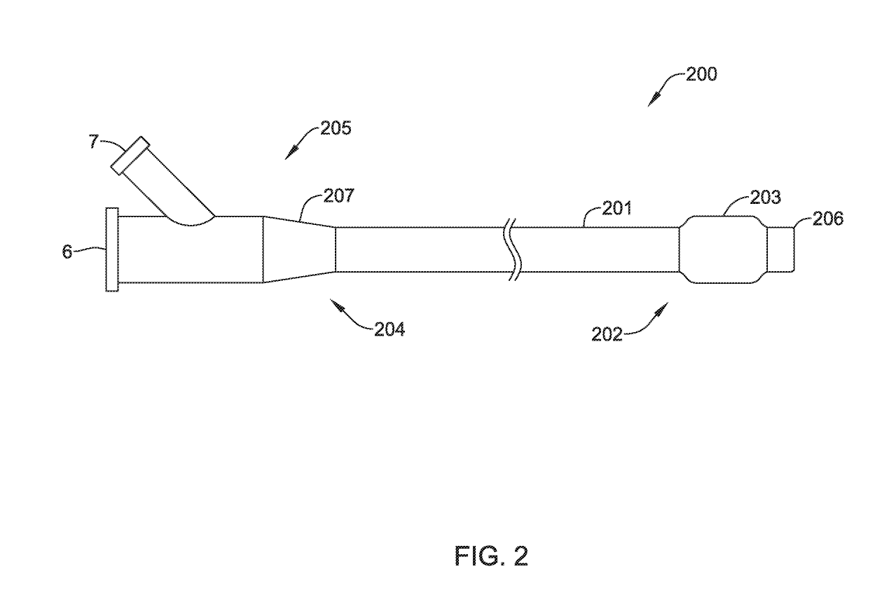

FIG. 2 depicts an example dilation device 200 that may be used in conjunction with inflatable scaffold member 101 for dilating and treating stricture 14 of FIG. 1. As seen in FIG. 2, dilation device 200 may comprise elongate member 201 and balloon member 203 connected to elongate member 201 near distal region 202. Dilation device 200 may additionally include manifold 205 connected to proximal region 204 of elongate member 201. The manifold may include hub 207 and/or other structures to facilitate connection to other medical devices (e.g., syringe, stopcocks, Y-adapter, etc.) and to provide access to one or more lumens defined within elongate member 201. In some cases, hub 207 may include ports 6 and/or 7, which provide individual access to one or more lumens extending through at least a portion of dilation device 200. In other cases, hub 207 may have a single port, two ports, or any other number of ports. Manifold 205 may also include a strain relief portion adjacent proximal region 18 of elongate member 201.

Although as described herein as being used to treat stricture 14 of esophagus 12, in other cases dilation device 200 may be used in many different applications. For instance, dilation device 200 may be used to dilate an artery or other blood vessel to maintain blood flow through the vessel. These are just a few example applications. Accordingly, dilation device 200, in different embodiments may have any of a number of different sizes and/or lengths which are appropriate for different applications. In some embodiments, elongate member 201 may be a catheter shaft. An exemplary catheter that may be utilized in accordance with the various embodiments as described herein is shown and described in U.S. Pat. No. 8,182,465, which is incorporated herein by reference in its entirety for all purposes.

As mentioned, elongate member 201 may define one or more lumens. Some example lumens that may extend through elongate member 201 include at least one guidewire lumen and one or more inflation lumens. Any lumens that do extend through elongate member 201 may terminate at or near distal region 202 of elongate member 201. For instance, one or more inflation lumens may open into an interior of balloon member 203. When included, a guidewire lumen may extend all the way through balloon member 203 and terminate at distal end 206.

Elongate member 201 may be made from any suitable biocompatible polymer—that is a polymer that is safe for use within a human body. Some suitable polymeric materials include, but are not necessarily limited to, polyamide, polyether block amide, polyethylene, polyethylene terephthalate, polypropylene, polyvinylchloride, polyurethane, polytetrafluoroethylene, polysulfone, and copolymers, blends, mixtures or combinations thereof.

Balloon member 203 may be an annular balloon disposed around distal region 202 of elongate member 201. In some embodiments, balloon member 203 may be comprised of one or more materials such as silicone, thermoplastic polyurethane (TPU), SIBS (poly styrene-isobutylene-styrene block copolymer), polyurethane, SEBS styrene ethylene butylene styrene block copolymer, other styrenic block copolymers, or other suitable materials. In at least some embodiments, balloon member 203 may be compliant, such that balloon member 203 stretches as more inflation media is delivered into balloon member 203. However, in other embodiments, balloon member 203 may be non-compliant and may have a static, defined volume.

In general, balloon member 203 may have an inflated configuration and an un-inflated configuration. To inflate balloon member 203, a user, such as a physician, may deliver inflation media to the one or more inflation lumens of elongate member 201, e.g. through one or more ports of manifold 205. Example inflation media include water, saline solution, and other biologically safe liquids. To deflate balloon member 203, the user may withdraw the inflation media from balloon member 203, for example by using a pump or vacuum or other suction device.

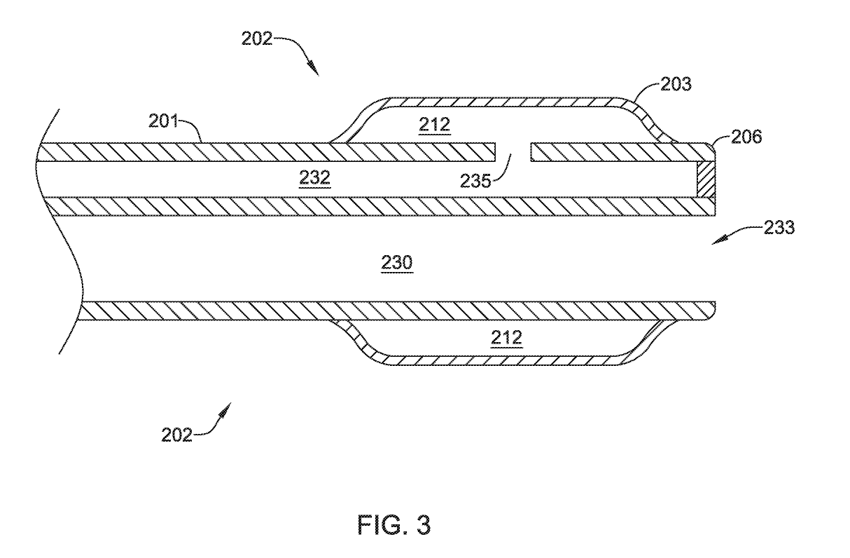

FIG. 3 depicts distal region 202 of dilation device 200 in partial cross-section with balloon member 203 in an un-inflated configuration. As seen in FIG. 3, elongate member 201 may include guidewire lumen 230 extending all the way through balloon member 203 and ending in guidewire port 233. FIG. 3 also depicts inflation lumen 232 that is in communication with the interior 212 of balloon member 203 by way of port 235

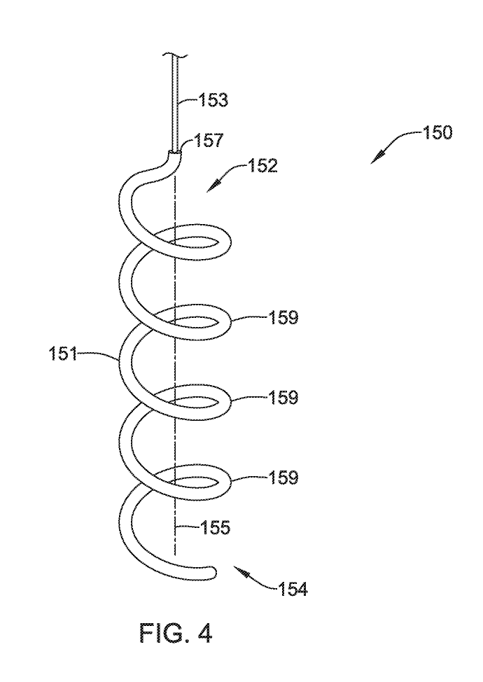

FIG. 4 depicts inflatable scaffold device 150. Inflatable scaffold device 150 includes both inflatable scaffold member 151 and inflation member 153. Inflatable scaffold member 151 in some embodiments may generally extend from a proximal end 152 to distal end 154 along longitudinal axis 155. In at least some embodiments, inflatable scaffold member 151 may be a shaped tubular member and may have a helical coil shape including a plurality of rings 159, as depicted in FIG. 4. Inflatable scaffold member 151 may be made from any suitable polymeric materials, such as those described with respect to elongate member 201 and balloon member 203. Inflatable scaffold member 151 may define a lumen that is continuous throughout inflatable scaffold member 151.

Inflation member 153 may also define a lumen that is in fluid communication with the lumen of inflatable scaffold member 151. FIG. 4 depicts the distal end of inflation member 153 connecting to proximal end 152 of inflatable scaffold member 151, at connection 157. In some embodiments, connection 157 may be a frangible connection. For instance, connection 157 may be a gated connection—that is the wall thickness of inflatable scaffold member 151 or inflation member 153 may be relatively thin compared to the wall thickness of inflatable scaffold member 151 or inflation member 153 at other points along the members 151, 153. In other embodiments, one or more frangible features, for example perforations through or recesses in the outer wall of inflatable scaffold member 151 or inflation member 153, may be used to create such a frangible connection. This frangible connection may be configured so that when a force is applied to inflation member 153 relative to inflatable scaffold member 151, the frangible connection breaks. In some instances, the relative force may be a retraction force on inflation member 153. In other embodiments, as will be described herein, inflation member 153 may be attached to balloon member 203. In such embodiments, the relative force causing connection 157 to break may be the deflation of balloon member 203.

As with dilation device 200, although as described herein as being used to treat stricture 14 of esophagus 12, in other cases inflatable scaffold device 150 may be used in many different applications. For instance, inflatable scaffold device 150 may be used to maintain an opening in an artery or other blood vessel to maintain blood flow through the vessel. These are just a few example applications. Accordingly, inflatable scaffold device 150, including inflatable scaffold member 151 and inflation member 153, in different embodiments may have any of a number of different sizes and/or lengths which are appropriate for different applications.

Generally, inflatable scaffold member 151 may have an inflated configuration and an un-inflated configuration. Accordingly, as the lumen of inflation member 153 is in fluid communication with the lumen of inflatable scaffold member 151, a user, such as a physician, may deliver inflation media to the lumen of inflation member 153 to inflate inflatable scaffold member 151. In different embodiments, different inflation media may be used to impart different physical properties to inflatable scaffold member 151, such as different stiffness, hardness, or strength of inflated inflatable scaffold member 151.

Some example inflation media include water, saline, one or more various contrast materials, different hardening epoxies, or one or more various foam-forming polymer materials. In embodiments where the inflation media comprises foam-forming polymer materials, the inflation media may comprise two or more separate reactants. Once the two or more separate reactants have mixed, the reactants may cure or harden into a solid polymer material or expanded foam, for example. In some embodiments, this foam-forming reaction may be aided by an application of heat, either from an external source or by the body of the patient, or electricity. In some cases, the two or more reactants may be mixed before being delivered inflation member 153. In other cases, however, the two or more separate reactants may be delivered through separate inflation lumens, for instance, inflation member 153 may define multiple inflation lumens, and all of the multiple inflation lumens open into the lumen of inflatable scaffold member 151. In some of these examples, the separate inflation media may only mix when entering inflatable scaffold member 151. In other such examples, separate inflation lumens may merge prior to opening into inflatable scaffold member 151. In some of these cases, inflation member 153 may include one or more mixing features where the multiple inflation lumens merge to aid in the mixing of the separate inflation media.

In embodiments where the inflation media comprises polymer material reactants which, when mixed, form a foam structure, the separate polymer material reactants may begin as liquids. Once the liquid reactants are mixed together, the liquid reactants may begin to expand in a foaming fashion and eventually harden or cure. As one example, the interior inflatable scaffold member 151 may be coated with a super absorbant polymer (SAP) such as lightly cross-linked poly sodium acrylate, a polyether block amide like PEBAX® MV1074 or Tecophilic® Lubrizol HP-60d, or other similar polymers. Then, water or saline may be delivered into the interior of inflatable scaffold member 151 through inflation member 153 to mix with the expanding or foam-forming polymer.

In other embodiments, the inflation media may comprise an aqueous solution (e.g. 1% solids) of polyacrylic acid which may be delivered into inflatable scaffold member 151 through a first inflation lumen of inflation member 153 and an aqueous solution of base (e.g. NaOH or sodium bicarbonate) which may be delivered through a second inflation lumen of inflation member 153. Mixing of the two solutions may result in neutralization of the polyacrylic acid and form gelled polysodium acrylate.

In still other embodiments, a foam may be formed using a reaction according to equation (1).

isocyanate+polyol+water=polyurethane+CO2=polyurethane foam (1)

Example isocyanates that may be used include hexamethyline diisocyanate (HDI), toluene diisocyanate (TDI), xylene diisocyanate, methylene diphenyl diisocyanate (MDI), lysine diisocyanate, and isophorone diisocyanate. Example polyols that may be used include polyether, polybutadiene polyols, polysiloxane polyols, polypropylene glycols (PPG), and polyethylene glycols (PEG). In still other examples, the inflation media may comprise polymethyl methacrylate (PMMA) or one or more rapid self curing silicones (e.g. Equinox® One to One Silicone).

Where inflation of inflatable scaffold member 151 includes more than one reactant, least one of the reactants may be predisposed within the lumen of inflatable scaffold member 151. For instance, in the above example, the lumen of inflatable scaffold member 151 may contain the isocyanate. Accordingly, when inflatable scaffold member 151 is to be inflated, a user may deliver a mixture of polyol and water into the lumen of inflatable scaffold member 151. The delivered media may then react with the isocyanate already disposed within the lumen of inflatable scaffold member 151 according to equation (1) to create a polyeurethane foam.

In general, by utilizing different reactants or reactants in varying proportions, foams or gelated materials having specific, differing properties may be formed. For instance, various foams used to inflate inflatable scaffold member 151 may have pore sizes ranging from 5-500 micrometers and may have anywhere between 10-10,000 cells. Further, the stiffness of the foam or gelated material may be controllable based on the types and quantities of the reactants used.

In at least some embodiments, when in the inflated configuration, inflatable scaffold member 151 may be generally radially non-compliant, with the radial direction extending outward perpendicularly from longitudinal axis 155. In some additional or alternative embodiments, inflatable scaffold member 151 may be generally flexible in the longitudinal direction extending generally parallel to longitudinal axis 155.

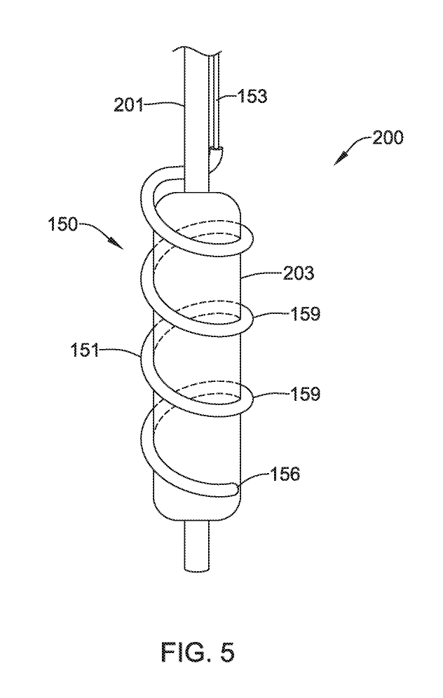

FIG. 5 depicts inflatable scaffold device 150 disposed around balloon member 203 of dilation device 200. As can be seen, inflatable scaffold member 151 may be wrapped around balloon member 203 in a helical fashion. In some embodiments, inflatable scaffold member 151 may be releasably coupled to balloon member 203 and/or elongate member 201. For instance, in at least some examples, inflatable scaffold member 151 may be secured to balloon member 203 at connection point 156. In these examples, having inflatable scaffold member 151 secured to balloon member 203 at least at connection point 156 may prevent inflatable scaffold member 151 from unravelling when inflated. In such examples, inflatable scaffold member 151 may be secured to balloon member 203 with a soluble adhesive. The adhesive may be soluble in water or saline. Accordingly, once inflatable scaffold member 151 has been positioned and inflated, a user may deliver the appropriate solvent to inflatable scaffold member 151 and balloon member 203. In other examples, the adhesive may be soluble in the aqueous environment of the body lumen where inflatable scaffold member 151 has been deployed.

In other examples, instead of securing inflatable scaffold member 151 to balloon member 203 with an adhesive, inflatable scaffold member 151 may include one or more longitudinal connecting members (not shown) connecting rings 159 together. The longitudinal connecting members may be conduits defining lumens that are a part of the lumen defined by inflatable scaffold member 151. In other embodiments, however, the longitudinal connecting members may be separate members added to inflatable scaffold member 151 to connect rings 159.

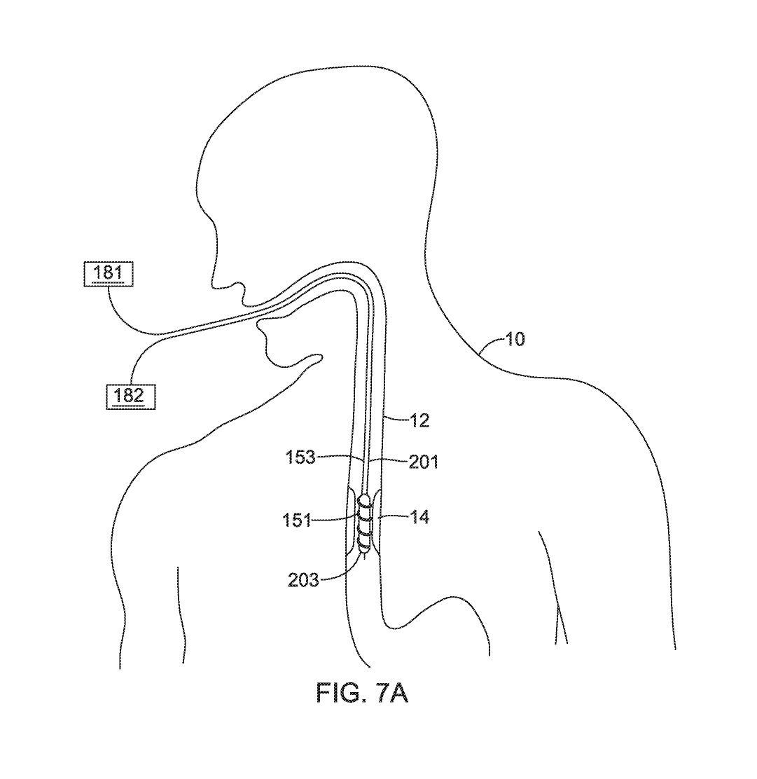

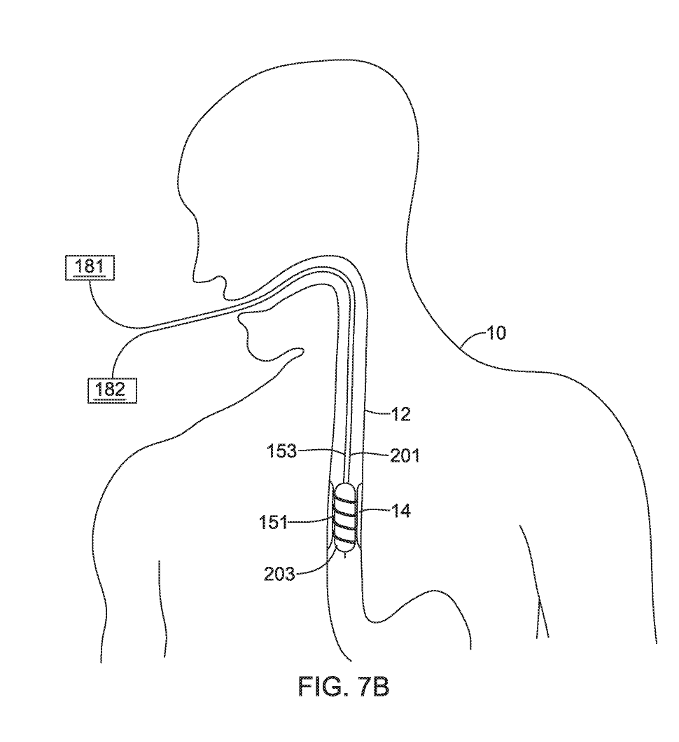

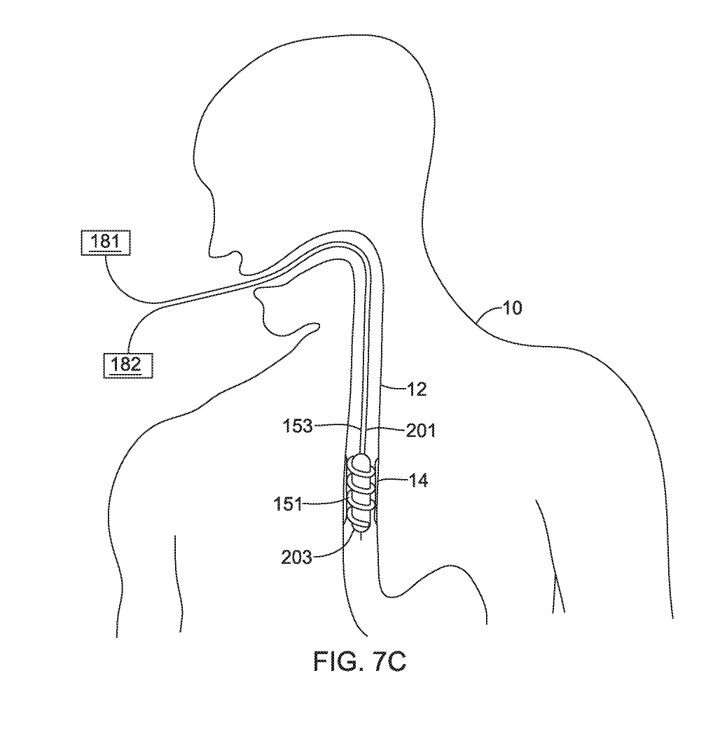

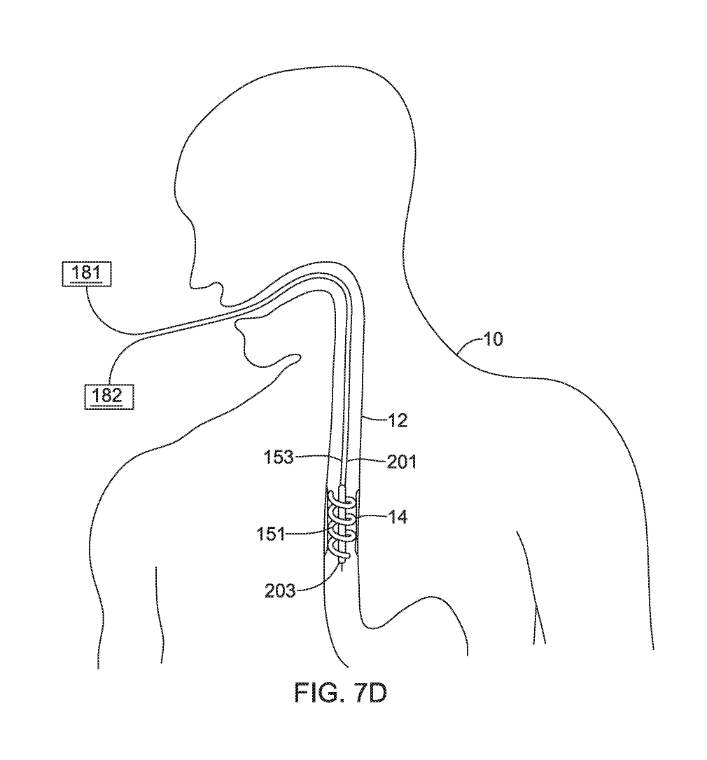

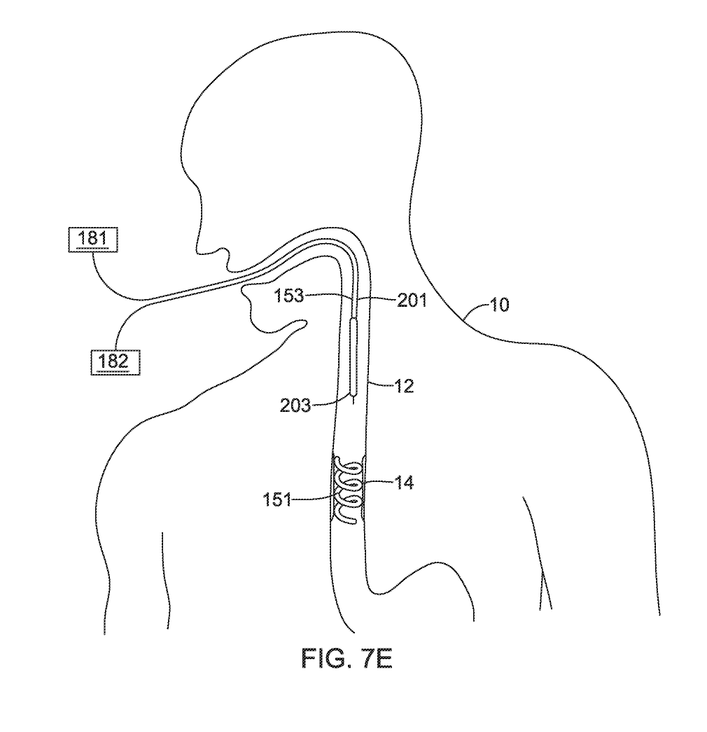

Additionally, as can be seen in FIG. 5, inflation member 153 may extend alongside elongate member 201. In some examples, inflation member 153 may be connected to elongate member 201 at one or more points along elongate member 201 by one or more connecting members (not shown) or through various bonding techniques. As will be described in more detail with respect to FIGS. 7A-7E, inflatable scaffold device 150 and dilation device 200 may be delivered to a stricture site in the configuration shown in FIG. 5.

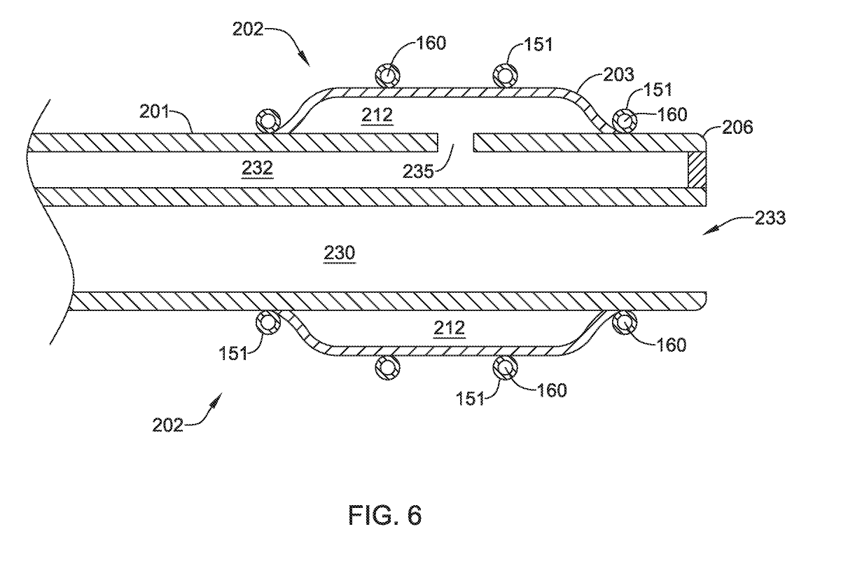

FIG. 6 depicts a partial cross-section of distal region of dilation device 200, with inflatable scaffold member 151 disposed around balloon member 203 and elongate member 201. In FIG. 6, lumen 160 of inflatable scaffold member 151 is clearly visible. Additionally, connection point 156 where inflatable scaffold member 151 connects to balloon member 203 and/or elongate member 201 is more easily visible than in FIG. 5.

FIGS. 7A-7E depict a procedure for dilating a stricture and implanting an inflatable scaffold member using the device described herein. For instance, FIG. 7A depicts patient 10, including esophagus 12 and stricture 14, as described with respect to FIG. 1. FIG. 7A also depicts dilation device 200 and inflatable scaffold member 151 disposed around balloon member 203. In the depicted example, elongate member 201 and inflation member 153 are both depicted as a single solid line, but it should be understood that each line represents the structures described previously, such as tubular members defining one or more lumens. FIG. 7A also depicts user interfaces 181 and 182 connected to proximal ends of elongate member 201 and inflatable scaffold member 151, respectively. In some examples, user interface 181 may include means for delivering inflation media to elongate member 201 and balloon member 203. In some additional or alternative embodiments, user interface 181 may additionally include a pressure monitor or sensor for monitoring the pressure inside of elongate member 201 and balloon member 203. In at least some embodiments, user interface 181 may further include a reservoir of inflation media for delivering to elongate member 201 and balloon member 203. User interface 182 may include similar elements as user interface 181, except user interface 182 may be connected to inflation member 153 and inflatable scaffold member 151.

As a first step, a user, such as a physician, may position balloon member 203 and inflatable scaffold member 151 within stricture 14, as is depicted in FIG. 7A. In some examples, dilation device 200 and inflatable scaffold device 150 may be sized to be used in conjunction with an endoscope. In such example, the user may be able to more easily see the operation of dilation device 200 and inflatable scaffold device 150.

Once in position, the user may deliver inflation media through elongate member 201 and into balloon member 203, thereby inflating balloon member 203, as depicted in FIG. 7B. Inflating balloon member 203 may dilate stricture 14 by pressing outward on stricture 14. In some embodiments, balloon member 203 may be non-compliant and may have an appropriate size chosen by the user. In other embodiments, however, balloon member 203 may be compliant. In such cases, the user may monitor the pressure inside balloon member 203 through user interface 181. The user may inflate balloon member 203 until the interior of balloon member 203 reaches a predetermined pressure.

After the user has inflated balloon member 203 to the appropriate size or pressure, the user may inflate inflatable scaffold member 151, as depicted in FIG. 7C. For instance, the user may deliver inflation media through inflation member 153 to inflatable scaffold member 151 by using user interface 182. In some embodiments, the inflation media may gel otherwise harden, thereby imparting some rigidity to inflatable scaffold member 151. This rigidity may operate to help maintain an opening through esophagus 12.

Once the inflation media has gelled or otherwise hardened, a user may deflate balloon member 203, as shown in FIG. 7D. For instance, user interface 181 may include a pump or other device for removing inflation media from balloon member 203. In these embodiments, where inflatable scaffold member 151 was releasably coupled to balloon member 203, the user may first decouple inflatable scaffold member 151 from balloon member 203. For instance, the user may introduce a solvent into esophagus 12 where inflatable scaffold member 151 was coupled to balloon member 203 by one or more adhesives. In other examples, deflating balloon member 203 may be enough to decouple inflatable scaffold member 151 from balloon member 203.

Once balloon member 203 has been deflated and decoupled from inflatable scaffold member 151, the user may remove dilation device 200 from within stricture 14 and patient 10, while leaving behind inflatable scaffold member 151. Additionally, as seen in FIG. 7E, inflation member 153 may also be removed along with dilation device 200. For example, inflation member 153 may have been decoupled from inflatable scaffold member 151 when balloon member 203 was deflated. Alternatively, as the user retracts dilation device 200, to which inflation member 153 is connected, there may be a relative force between inflatable scaffold member 151 and inflation member 153, as inflatable scaffold member 151 may be held in place within stricture 14 by friction as inflatable scaffold member 151 may press radially outward on stricture 14 after inflation. This relative force between inflatable scaffold member 151 and inflation member 153 may be enough to separate inflation member 153 from inflatable scaffold member 151, for example by breaking the connection between inflatable scaffold member 151 and inflation member 153.

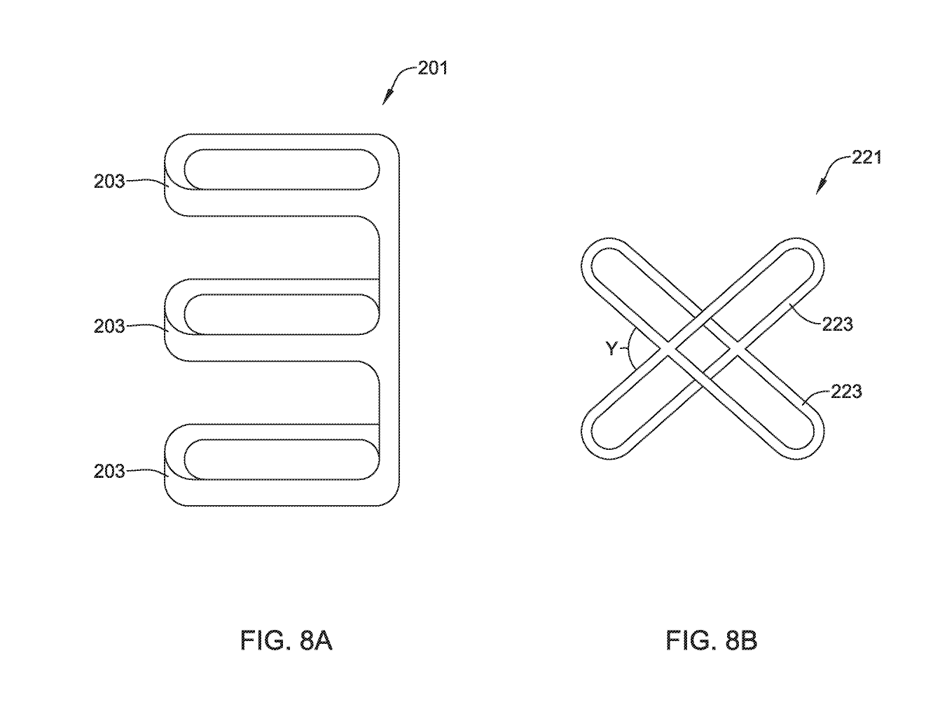

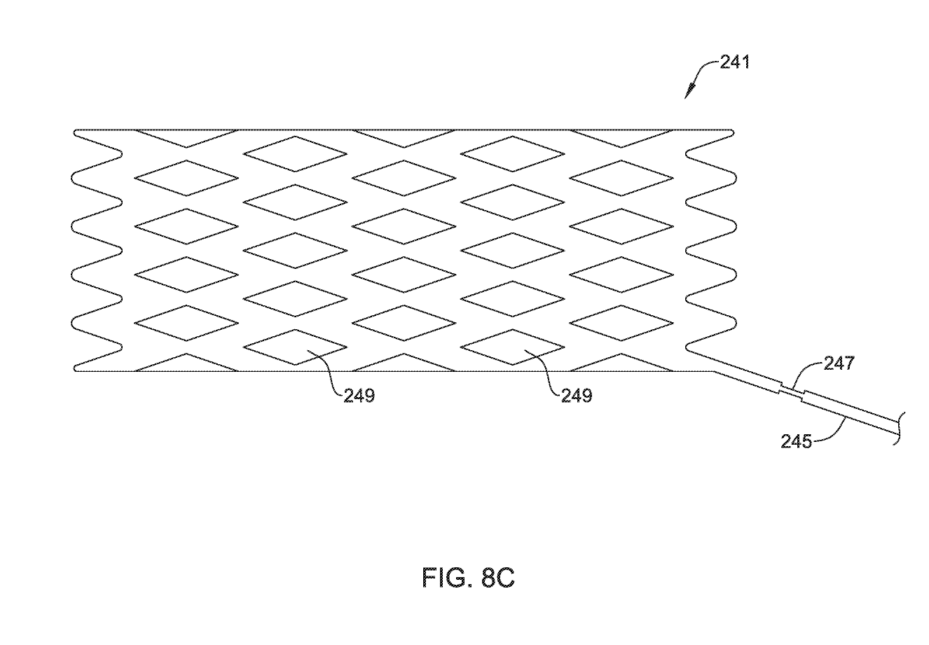

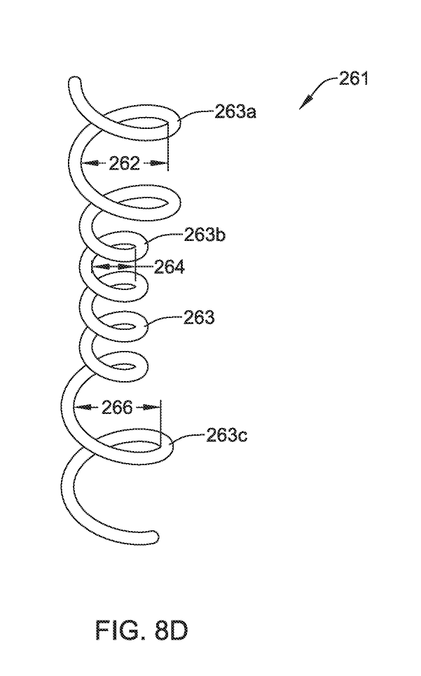

FIGS. 8A-8D all depict alternative inflatable scaffold members. For example, FIG. 8A depicts inflatable scaffold member 201. Inflatable scaffold member 201 comprises a series rings 203, similar to inflatable scaffold member 151. However, in the example of inflatable scaffold member 201, rings 203 are disposed in a concentric manner. Although only depicted as three concentric rings, in other embodiments, inflatable scaffold member 201 may have any suitable number of rings 203 in order to span a stricture. As with inflatable scaffold member 151, inflatable scaffold member 201 may define a single continuous lumen. Although not shown, an inflation member may connect to any portion of inflatable scaffold member 201, for example through a frangible connection as described with respect to inflatable scaffold member 151. As inflatable scaffold member 201 may define a single, continuous lumen, regardless of where the inflation member connects to inflatable scaffold member 201, delivering inflation media through the inflation member may fill the entirety of inflatable scaffold member 201.

FIG. 8B depicts another alternative inflatable scaffold design. Inflatable scaffold member 221 may also comprise a series of rings 223. However, in the example of inflatable scaffold member 221, rings 223 may not be concentric rings, as in inflatable scaffold member 201, or form a helical coil, as in inflatable scaffold member 151. Rather, rings 223 of inflatable scaffold member 221 may be connected at an angle, angle Y. Angle Y may generally range from five degrees to eighty-five degrees, or from twenty degrees to sixty degrees, and in some specific embodiments may be twenty-five degrees, forty-five degree, or sixty-five degrees. The specific angle between rings 223 of the particular inflatable scaffold member 221 used may depend on the specific application or geometry of the stricture. Additionally, in other embodiments, inflatable scaffold member 221 may include additional numbers of rings 223. For instance, inflatable scaffold member 201 may include three, four, five, or any other suitable number of rings 223. In a similar manner to that depicted in FIG. 8B, all of rings 223 may be connected at an angle, whether through the same connection point, or through difference connection points between different rings 223, and the angles at each of the connection points may differ. In at least some embodiments, rings 223 may be interconnected to form a single continuous lumens. Additionally, although not shown in FIG. 8B, an inflation member may connect to any portion of inflatable scaffold member 221, for example through a frangible connection as described with respect to inflatable scaffold member 151. As inflatable scaffold member 221 may define a single, continuous lumen, regardless of where the inflation member connects to inflatable scaffold member 221, delivering inflation media through the inflation member may fill the entirety of inflatable scaffold member 221.

FIG. 8C depicts yet another alternative inflatable scaffold, inflatable scaffold member 241. Instead of including a number of rings, as in the inflatable scaffold members of FIGS. 4 and 8A-8B, inflatable scaffold member 241 may be a mesh-like structure or network. For instance, inflatable scaffold member 241 may be an inflatable tubular structure including a number of apertures 249 disposed throughout inflatable scaffold member 241. In at least some embodiments, inflatable scaffold member 241 may resemble a traditional woven stent in shape. Inflatable scaffold member 241 may also define a single continuous lumen. FIG. 8C also depicts inflation member 245 connected to inflatable scaffold member 241 through connection 247, which in some examples may be a frangible connection. Accordingly, as inflatable scaffold member 241 may define a single, continuous lumen, delivering inflation media through inflation member 245 may fill the entirety of inflatable scaffold member 241.

Although shown disposed proximate an end of inflatable scaffold member 241, in other embodiments, inflation member 245 may connect to inflatable scaffold member 241 at any point on inflatable scaffold member 241.

FIG. 8D is yet another depiction of an example inflatable scaffold member. For instance, FIG. 8D depicts inflatable scaffold member 261. Inflatable scaffold member 261 may be similar to inflatable scaffold member 151 in that inflatable scaffold member 261 may have a helical coil shape made from rings 263. However, as shown in the example of FIG. 8D, inflatable scaffold member 261 may have rings 263 with different diameters. For instance, a first ring 263a may have a first diameter 262, while a second ring 263b may have a second diameter that is different than the first diameter 262. In some examples, second diameter 264 may be smaller than first diameter 262, however, in other examples the reverse may be true. Additionally, inflatable scaffold member 261 may have third ring 263c with a third diameter 266. In some embodiments, third diameter may be different than both first diameter 262 and second diameter 264. However, in at least some embodiments, third diameter 264 may be similar to or the same as first diameter 262. For instance, first diameter 262 and third diameter 266 may be similar and generally greater than second diameter 264. Inflatable scaffold member 261 having end rings with larger diameters than intermediate rings may help hold inflatable scaffold member 261 in place within a stricture. However, in other examples, first and third diameters 262, 266 may be different, yet both may still be greater than second diameter 264.

Additionally, although only described with respect to FIG. 8D, the other embodiments of inflatable scaffold members described herein may include rings or portions with varying diameters. For instance, rings 203 and 223 of inflatable scaffold members 201, 221 may have different diameters as described with respect to rings 263 of FIG. 8D. Additionally, in at least some examples, inflatable scaffold member 241 may have different portions with different diameters. For instance, both end portions of inflatable scaffold member 241 may have larger diameters than an intermediate portion of inflatable scaffold member 241. This may give inflatable scaffold member 241 a generally hourglass shape. However, in other embodiments, inflatable scaffold member 241 may only include one end portion with a greater diameter than the rest of inflatable scaffold member 241.

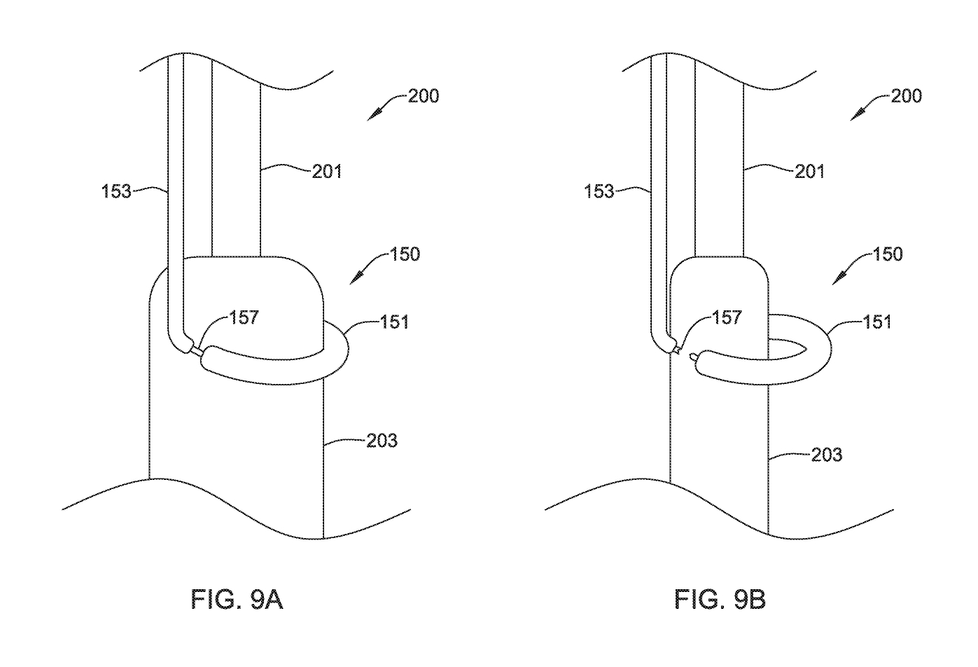

FIGS. 9A and 9B depict a close-up of inflatable scaffold member 151 disposed around balloon member 203 when balloon member 203 is in the inflated configuration and in the deflated configuration. FIG. 9A depicts both inflatable scaffold member 151 and balloon member 203 in their inflated configurations. For instance, FIG. 9A shows an example of how dilation device 200 and inflatable scaffold device 150 may be configured, with both inflatable scaffold member 151 and balloon member 203 in their inflated configurations, after having been positioned within a stricture. As described previously, inflation member 153 may be connected to balloon member 203, and in some particular examples inflation member 153 may be connected to balloon member proximate connection 157. Once inflatable scaffold member 151 has been inflated, balloon member 203 may then be deflated in preparation for removal of dilation device 200 and inflation member 153 from the patient.

FIG. 9B shows the configuration of dilation device 200 and inflatable scaffold device 150 after balloon member 203 has been deflated. In at least some examples, connection 157 may be a frangible connection, as described previously. In such instances, deflation of balloon member 203, to which inflation member 153 is attached proximate connection 157, may cause connection 157 to break, as shown in FIG. 9B. For instance, inflatable scaffold member 151 may have been inflated with one or more hardening or curable agents, which imparted a rigidity to inflatable scaffold member 151. In such examples, as balloon member 203 is deflated, inflatable scaffold member 151 may maintain its shape. The deflation of balloon member 203, then, may pull inflation member 153 away from inflatable scaffold member 151. This relative movement may be sufficient to break connection 157, again as shown in FIG. 9B.

Although only shown with respect to inflatable scaffold member 151, the other inflatable scaffold members described herein may have a similar frangible connection to an inflation member. Accordingly, in at least some of those other inflatable scaffold member embodiments, the frangible connection may be broken upon deflation of a balloon member.

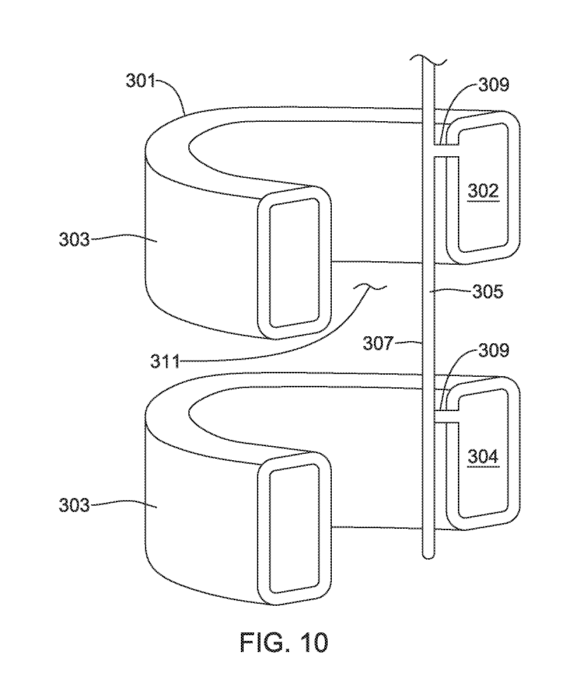

FIG. 10 depicts another example relation between an inflatable scaffold member 301 and an inflation member 305. FIG. 10 depicts an example where inflatable scaffold member 301 comprises a series of concentric rings 303. In the example of FIG. 10, when inflatable scaffold member 301 is disposed on a dilation device (not shown), a balloon member of the dilation device (not shown) may be disposed through opening 311 extending through rings 303. In at least some of these embodiments, inflation member 305 may be connected to the balloon member, for instance along longitudinal surface 307. Once the dilation device and inflatable scaffold member 301 have been disposed at an appropriate location, the balloon member and inflatable scaffold member 301 may be inflated. For example, inflation media may be delivered to lumens 302 and 304 of rings 303 through inflation ports 309. Similarly to connection 157 described previously, inflation ports 309 may connect inflation member 305 to rings 303 in a frangible manner. Once the balloon member and inflatable scaffold member 301 have been inflated, the balloon member may be deflated in preparation for removal of the dilation device and inflation member 305 from the patient. As inflation member 305 may be connected to the balloon member, deflation of the balloon member may pull inflation member 305 away from inflated inflatable scaffold member 301. This relative movement may be enough to cause inflation ports 309 to break, thereby severing the connection between inflatable scaffold member 301 and inflation member 305. Although, in other examples, inflation ports 309 may break their connection with inflatable scaffold member 301 once the dilation device is retracted to withdraw the dilation device out of the patient.

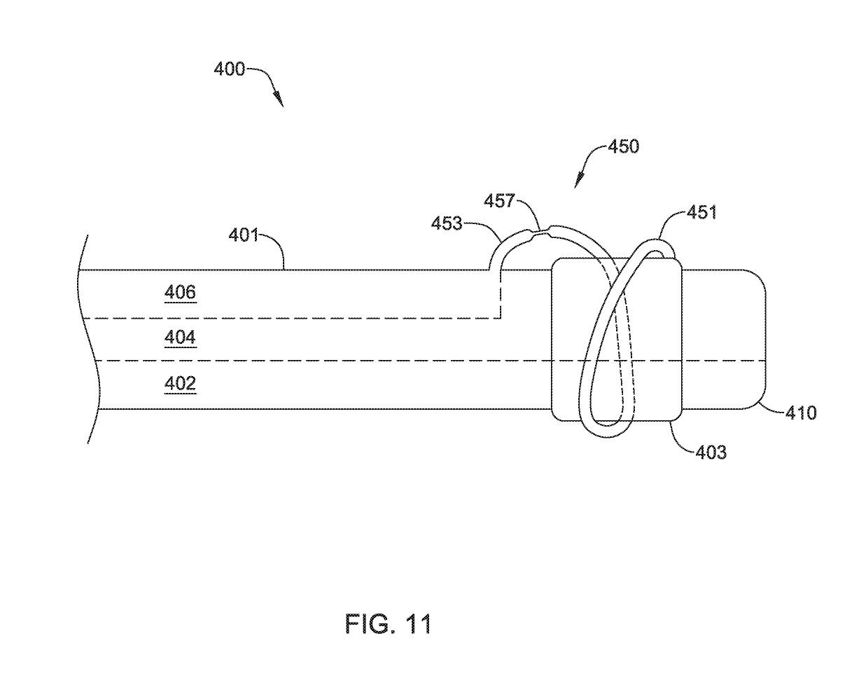

FIG. 11 depicts an alternative dilation device and inflatable scaffold device, dilation device 400 and inflatable scaffold device 450. In the example of FIG. 11, dilation device 400 includes elongate member 401 and balloon member 403, and inflatable scaffold device 450 includes inflatable scaffold member 451 connected to inflation member 453 through connection 457. Each of these components may be similar to similarly named components described with respect to FIGS. 2, 4, and 5. In the example of FIG. 11, however, instead of inflation member 453 extending alongside elongate member 401, inflation member 453 connects to elongate member 401 and is in fluid communication with a lumen of elongate member 401.

For example, elongate member 401 may define a number of lumens, including lumens 402, 404, and 406, as depicted in FIG. 11. One lumen, for instance lumen 402, may be a guidewire lumen that extends all the way through balloon member 403 terminating at distal end 410 of elongate member 401. Another of the lumens, for instance lumen 404, may be an inflation lumen that opens into the interior of balloon member 403, for instance through one or more inflation ports (not shown). A user may deliver inflation media through inflation lumen 404 to inflate balloon member 403. Another lumen, for instance lumen 406, may also be an inflation lumen. However, inflation lumen 406 may terminate before balloon member 403 and connect to inflation member 453 such that inflation lumen 403 is continuous with the lumen defined by inflation member 453. Accordingly, a user may deliver inflation media to inflation lumen 406 in order to inflate inflatable scaffold member 451. In some alternative embodiments of FIG. 11, inflatable scaffold device 450 may not include inflation member 453. Instead, inflation lumen 406 may connect directly to inflatable scaffold member 451 through connection 457, which is connected directly to elongate member 401. Additionally, although described only with respect to FIG. 11, elongate members of other embodiments described herein may contain an inflation lumen in fluid communication with the lumen of an inflatable scaffold member, either through an inflation member, or directly through a connection such as connection 457.

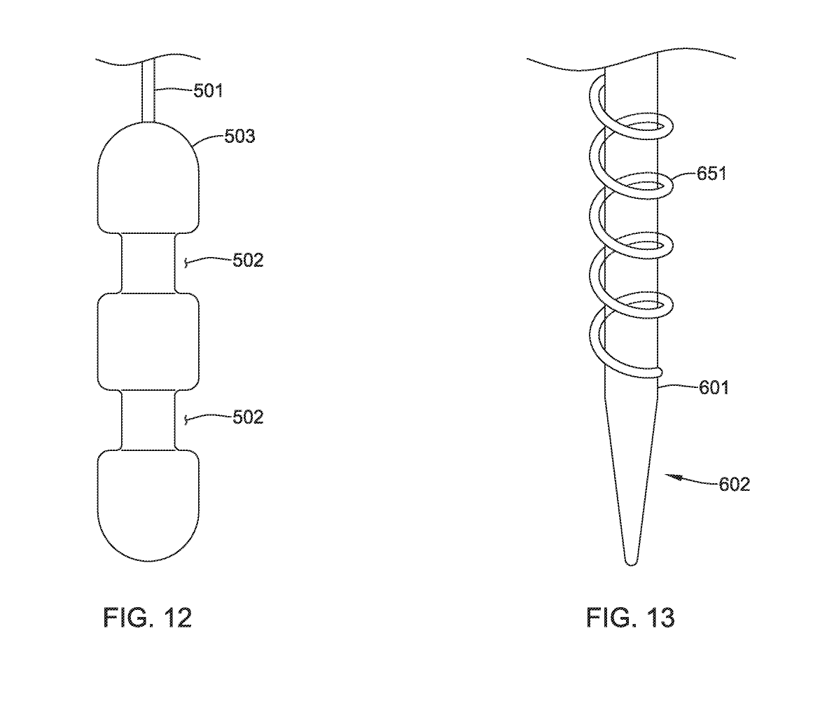

FIG. 12 depicts an alternative balloon member, balloon member 503. As shown in FIG. 12, balloon member may be connected to elongate member 501, in a similar manner to previously described elongate members and balloon members. However, balloon member 501 may include one or more recessed portions. Balloon member 503 may be generally non-compliant and be formed with recesses 502. In some embodiments, recesses 502 may be formed with a shape configured to hold an inflatable scaffold member. For instance, in the example of FIG. 12, recesses 502 of balloon member 503 may be configured to hold an inflatable scaffold member comprising a pair of concentric rings. However, in other embodiments, recesses 502 may be shaped to hold other shaped inflatable scaffold members, such as any of those described herein. For instance, recesses 502 may be shaped into a helical coil, or may include a number of crossed recesses to hold inflatable scaffold member 241. In these examples, recesses 502 may aid in inflation of the inflatable scaffold member. When balloon member 503 is inflated within a stricture, balloon member 503 may impart a force on the stricture. This force may make it difficult to deliver inflation media to the inflatable scaffold member to inflate the inflatable scaffold member. Accordingly, recesses 502 may provide an area where balloon member 503 does not press on the stricture, or presses on the stricture with less force than other areas of balloon member 503. This reduced force may allow inflation media to be delivered to the inflatable scaffold member and for the inflatable scaffold member to become inflated. It should be understood any of the balloon members described herein may include one or more recesses 502 as described with respect to balloon member 503.

In at least some embodiments where balloon member includes one or more recesses 502, the inflatable scaffold device may not include an inflatable scaffold member. Instead, one or more reactants may be disposed within the one or more recesses. Once balloon member 503 is in place, one or more additional reactants may be introduced into the body lumen. The one or more additional reactants may react with the one or more reactants disposed within recesses 502 to form an expanding foam or a curable epoxy, or react to form another hardened material. After introduction of the one or more additional reactants, balloon member 503 may be inflated and expand against the wall of the body lumen or stricture. When balloon member 503 is inflated, the reaction may continue to form a hardened material within recesses 502. Once the reaction has finished, balloon member 503 may be deflated, leaving the hardened material in place in the shape of recesses 502 and maintaining an opening through the dilated body lumen or stricture. In other embodiments, the one or more additional reactants may be introduced into the body lumen after balloon member 503 has been inflated.

FIG. 13 depicts an alternative dilation device, dilation device 601, which does not include a balloon member. Dilation device 601 may be a rigid, or semi-rigid, device such as a bougie or other medical device. Dilation device 601 may additionally include a tapered region 602 where dilation device 601 tapers. The taper of dilation device 601 may aid in insertion of dilation device 601 into a body lumen and also in dilation of a stricture of the body lumen. For instance, instead of simply including two portions with different diameters, tapered region 602 may help to slowly widen the stricture as dilation device 601 is advanced through the lumen. Inflatable scaffold member 651 may be disposed around dilation device 601 where dilation device 601 has a diameter that is the desired diameter of the body lumen after dilation. Once dilation device 601 has been inserted into the body lumen and dilated the stricture to the desired size, inflatable scaffold member 651 may be in position within the stricture. Accordingly, inflation media may then be delivered to inflatable scaffold member 651 to inflate inflatable scaffold member 651. Once inflated, dilation device 601 may be retracted to withdraw dilation device 601 from the patient. Although not shown in FIG. 13, an inflation member used to inflate inflatable scaffold member 651 may be disconnected from inflatable scaffold member 651 and withdrawn along with dilation device 601, according to one or more techniques described herein with respect to other figures. In still other embodiments, the dilation device may be similar to devices such as a transition obturator or expanding obturator.

Those skilled in the art will recognize that aspects of the present disclosure may be manifested in a variety of forms other than the specific embodiments described and contemplated herein. Additionally, although various features may have only been described in conjunction with a particular Figure or embodiment, each feature described with respect to each embodiment may be combined with each other feature described herein in other contemplated embodiments. For instance, some features may have been only described with respect to dilation device 200. However, at least some contemplated embodiments of dilation devices 300 and/or 400 include the features exclusively detailed with respect to dilation device 200. Accordingly, departure in form and detail may be made without departing from the scope and spirit of the present disclosure as described in the appended claims.

What is claimed is:

1. A device for dilating a lumen of a body, the device comprising:
an elongate member having a proximal end and a distal end;
an inflatable scaffold member disposed on the distal end of the elongate member, the inflatable scaffold member defining a lumen;
an inflation member in fluid communication with the lumen of the inflatable scaffold member,
wherein the inflatable scaffold member is frangibly connected to the inflation member.

2. The device of claim 1, wherein the elongate member is a catheter shaft.

3. The device of claim 2, further comprising a balloon member disposed on the proximal end of the catheter shaft, wherein the inflatable scaffold member is disposed around the balloon member.

4. The device of claim 3, wherein the catheter shaft defines at least a guidewire lumen and a first inflation lumen, the first inflation lumen in fluid communication with an interior of the balloon member.

5. The device of claim 3, wherein the balloon member has a deflated configuration and an inflated configuration, wherein the frangible connection is configured to break when the balloon member transitions from the inflated configuration to the deflated configuration.

6. The device of claim 3, wherein the inflatable scaffold member is releasably connected to the balloon member.

7. The device of claim 1, wherein the inflatable scaffold member has an inflated configuration and a deflated configuration.

8. The device of claim 7, wherein in the inflated configuration, the inflatable scaffold member is radially non-compliant.

9. The device of claim 7, wherein the inflatable scaffold member extends from a proximal end to a distal end along a longitudinal axis, and wherein in the inflated configuration, the inflatable scaffold member is flexible in a longitudinal direction.

10. The device of claim 1, wherein the inflatable scaffold member is connected to the inflation member through a gated port.

11. The device of claim 1, wherein the inflatable scaffold member is releasably coupled to the elongate member.

12. The device of claim 1, wherein the inflatable scaffold member has a first diameter along a first section, and the inflatable scaffold member has a second diameter along a second section, wherein the first diameter is different than the second diameter.

13. An inflatable scaffold device, comprising:
a shaped tubular member defining a lumen; and
an inflation member defining an inflation lumen, the inflation lumen in fluid communication with the lumen of the shaped tubular member,
wherein the inflation member is connected to the shaped tubular member by one or more frangible inflation ports.

14. The inflatable scaffold device of claim 13, wherein the shaped tubular member has a helical coil shape.

15. The inflatable scaffold device of claim 13, wherein the shaped tubular member comprises a plurality of interconnecting conduit member forming a mesh network.

16. The inflatable scaffold device of claim 13, wherein the shaped tubular member has an inflated configuration and a deflated configuration.

17. The inflatable scaffold device of claim 16, wherein in the inflated configuration, the shaped tubular member is radially non-compliant.

18. A method for dilating a lumen of a body, the method comprising:
positioning an elongate member having a proximal end and a distal end within a stricture of a lumen, wherein the elongate member includes an inflatable scaffold member disposed on the distal end of the elongate member, the inflatable scaffold member defining a lumen and frangibly connected to an inflation member;
dilating the stricture with the elongate member;
inflating the inflatable scaffold member by delivering inflation media into the lumen of the inflatable scaffold member through the inflation member; and
removing the elongate member from within the stricture while leaving the inflatable scaffold member disposed within the stricture.

19. The method of claim 18, wherein inflating the inflatable scaffold member comprises inflating the inflatable scaffold member with one or more solidifying agents.

20. The method of claim 18, wherein:
the elongate member defines at least a first inflation lumen,
the elongate member includes a balloon member disposed on the distal end of the elongate member,
an interior of the balloon member is in fluid communication with the first inflation lumen, and
dilating the stricture with the elongate member comprises inflating the balloon member.
